(19) United States
(12) Patent Application Publication
(10) Pub. No.: US 2022/0151559 A1
(43) Pub. Date: May 19, 2022

(54) DEVICE FOR EXAMINING A CAPILLARY AREA

(71) Applicants: UNIVERSITE DE BORDEAUX, BORDEAUX (FR); CENTRE HOSPITALIER UNIVERSITAIRE DE BORDEAUX, TALENCE (FR); CENTRE TECHNOLOGIQUE ALPHANOV, TALENCE (FR)

(72) Inventors: Marie-Elise TRUCHETET, BORDEAUX (FR); Raphaël DEVILLARD, BORDEAUX (FR); Emmanuel CHALUMEAU, MERIGNAC (FR); Patrick MOREAU, BORDEAUX (FR); Matthieu POMMIES, PESSAC (FR)

(21) Appl. No.: 17/438,838

(22) PCT Filed: Mar. 13, 2020
(86) PCT No.: PCT/FR2020/050533

§ 371 Date: Sep. 13, 2021

(30) Foreign Application Priority Data
Mar. 14, 2019 (FR) 19 02599

(51) Int. Cl. A61B 5/00; A61B 5/1455; G02B 21/06; G02B 21/26

(52) U.S. Cl. CPC A61B 5/6826; A61B 5/0062; A61B 5/1455; A61B 5/0071; G02B 21/06; G02B 21/26; A61B 5/449

(57) ABSTRACT

The present disclosure concerns a device for examining a capillary area of the skin of a finger, said device comprising:
an optical microscope); —a light source for illuminating the capillary area; —a support comprising a recess for receiving the finger, said recess comprising an opening positioned opposite the microscope; —the microscope being mounted to rotate on the support about a main axis of the finger so as to scan the capillary area from one edge of the finger to the other; —immobilisation means being configured to adjust the dimension of the recess relative to the shape of the finger in order to immobilise the finger in the recess without exerting pressure on the finger. The examination device of the present disclosure is particularly suitable for examining a capillary area of the skin of a finger in order to detect vascular anomalies.

### CROSS-REFERENCE TO RELATED APPLICATIONS

This application is a National Stage of International Application No. PCT/FR2020/050533, having an International Filing Date of 13 Mar. 2020, which designated the United States of America, and which International Application was published under PCT Article 21(2) as WO Publication No. 2020/183116 A1, which claims priority from and the benefit of French Patent Application No. 1902599, filed on 14 Mar. 2019, the disclosures of which are incorporated herein by reference in their entireties.

### BACKGROUND

### Field

The present disclosure relates to a device for examining an area of the skin of a finger comprising blood capillaries, more specifically a device for examination by capillaroscopy, permitting in-depth analysis of the capillaries in order to detect vascular anomalies.

### Brief Description of Related Developments

Capillaries are small blood vessels which can be analyzed in particular in the area of the skin around the nail. The skin is in fact very thin in the periungual region at the nail cuticle. A capillaroscope is an instrument that allows physicians to perform examination of the capillaries, also referred to as capillaroscopy. The capillaroscope makes it possible to examine a number of parameters, such as visualizing the morphology of the capillaries, the capillary density at the periphery of the nail, the presence of edema or hemorrhages, avascular zones or an architectural disorganization. At the end of this examination, the physicians can make a diagnosis in order to establish in particular whether there is a vascular anomaly associated with the secondary Raynaud phenomenon, which is a first manifestation of scleroderma.

At present, vascular anomalies are analyzed at the end of the chain, in most cases in hospital, since only specialized physicians are equipped with a capillaroscope permitting the examination. The device generally comprises a frame on which are mounted the microscope, the light source, and a stage on which the patient places their finger opposite the exit of the optical objective of the microscope.

In the context of preventive medicine, very early management of patients suffering from scleroderma would make it possible to overcome the ineffectiveness of the therapeutic treatments that are currently offered. It is therefore necessary to have available a device permitting simple and rapid analysis of a capillary area, and thus in particular to screen patients at the pre-scleroderma stage in order to be able to offer suitable treatments and to prevent the progression of their condition.

Current examining devices are bulky, that is to say they take up a large volume and are difficult to carry. In addition, the cost of the equipment makes its acquisition difficult for a general practitioner. Thus, the examination of vascular anomalies is most often performed in hospital, hence often at the end of the care chain when the patient is already in an advanced phase of the disease. There is therefore a great need to generalize the capillary examination, in particular for the screening of abnormal Raynaud phenomena.

Current examining devices comprise a support in the form of a plate on which the patient's hand rests. The tip of the finger to be examined, placed under the microscope, is not blocked and can move in relation to the axis of observation of the microscope, and this can therefore adversely affect the quality of the image and make diagnosis difficult. Image quality is in fact essential for physicians to make a diagnosis and in particular to differentiate between a primary Raynaud phenomenon and a secondary Raynaud phenomenon and therefore detect an early stage of scleroderma. Thus, the use of a capillaroscope currently requires great expertise on the part of the physician and a long examination time.

There are portable capillaroscopes equipped with a movable microscope held by the physician, who moves one end of the microscope over the patient's finger from one edge of the cuticle to the other in order to examine the surface of the cuticle.

Such a device is generally very difficult to use because the physician has to keep the microscope in contact with the patient's finger, without moving it during viewing, while at the same time keeping the patient's finger in position during the examination. In addition, the surface of the capillary area to be examined, namely the surface of the cuticle, has a slight curvature. Under these conditions, when the physician moves the microscope or the patient's finger in order to perform the examination from one edge of the cuticle to the other, it is necessary to focus the microscope because of the loss of focus associated with the variation in the optical distance between the optical means of the microscope and the point of observation of the surface of the examination area due to the presence of the curvature. It is therefore not possible to take good-quality images from one edge of the cuticle to the other edge, since it is impossible for the physician to move the microscope with precision on the finger and bring the microscope into focus using just one hand. In addition, these devices have very reduced depths of focus, and the quality of the images obtained is generally not very good. In addition, the optical system provided in existing capillaroscopes does not correct for parasitic reflections that can adversely affect the quality of the images.

The present disclosure aims to remedy the drawbacks of the prior art described above.

The aim of the present disclosure is to make available a portable examining device which can be produced cost-effectively and allows the physician to perform the focusing of the microscope and the angular movement of the microscope relative to the observation surface using just one hand, while at the same time permitting immobilization of the finger and offering high-quality images with a spatial resolution of between 2 and 15 μm.

### SUMMARY

A device for examining a capillary area of the skin of a finger is proposed, said device comprising:
an optical microscope;
a light source for illuminating the capillary area;
a support comprising a recess for receiving said finger, said recess comprising an opening positioned opposite the microscope;
said microscope being mounted to rotate on the support about a main axis of the finger, so as to scan the capillary area from one edge of said finger to the other;
immobilizing means being configured to adjust the dimension of the recess relative to the shape of the finger, in order to immobilize the finger in said recess without exerting pressure on the finger.

According to an advantageous aspect of the present disclosure, the immobilizing means are configured to immobilize said finger in the recess without compressing it, in particular without compressing it in the three directions X, Y and Z.

In the present application, the immobilizing means make it possible to prevent the finger from moving in the recess, without exerting pressure on the finger, pressure that would cause a deformation of the skin of the finger.

By virtue of the immobilizing means, the present disclosure makes it possible to immobilize the finger, and thus immobilize the observation area, by avoiding uncontrolled movements of the patient, the immobilization being effected in the three directions X, Y and Z, without exerting pressure on the skin of the finger, which contributes to obtaining quality images.

Moreover, adjusting the dimension as close as possible to the anatomical dimension of the finger makes it possible to avoid possible compression of the blood capillaries, which could lead to diagnostic errors.

The features set out in the following paragraphs can be optionally implemented. They can be implemented independently of one another or in combination with one another:

The immobilizing means are configured to immobilize said finger by its upper and lower surfaces and by its side surfaces.

The microscope is mounted on an arm, one end of said arm being mounted to rotate on the support about the main axis of the finger, in such a way that the microscope can perform a scan of the capillary area from one edge of said finger to the other.

The support comprises a fixed base having a cavity, a movable component arranged in the cavity, said movable component being adapted to be moved between a first position, in which it forms within the space of the cavity a recess having a dimension adapted for insertion of the finger, and a second position, in which it forms within the space of the cavity a recess having a dimension adapted to immobilize the finger in the recess without exerting pressure on the finger.

The recess has a surface intended to match the shape of the finger when the finger is immobilized in the recess.

According to one aspect of the present disclosure, the device further comprises a stop element arranged in the recess in such a way as to form a bearing surface for one end of the finger along the main axis when the finger is positioned in the recess.

Advantageously, the light source is arranged to provide low-angled illumination on the capillary area.

According to one aspect of the present disclosure, the device further comprises a polarizer and an analyzer which are arranged on the optical path between the light source and the microscope in order to eliminate parasitic reflections.

According to another aspect, the device further comprises at least one optical band-pass filter intended to eliminate a red component of the image obtained by the microscope.

### BRIEF DESCRIPTION OF THE DRAWINGS

Other features, details and advantages of the present disclosure will become apparent from reading the following detailed description and from analyzing the accompanying drawings, in which:

FIG. 1

[FIG. 1] is a perspective view of a device for examining a capillary area of a finger according to one aspect of the present disclosure;

FIG. 2

[FIG. 2] is an enlarged perspective view of the support of the examining device of FIG. 1;

FIG. 3

[FIG. 3] is a sectional view of the device of FIG. 1 along a sectional plane A-A of FIG. 1;

FIG. 4

[FIG. 4] is a perspective view from the rear face of the device of FIG. 1;

FIG. 5

[FIG. 5] is an enlarged view of the upper plate with the light source of the device of FIG. 1;

FIG. 6

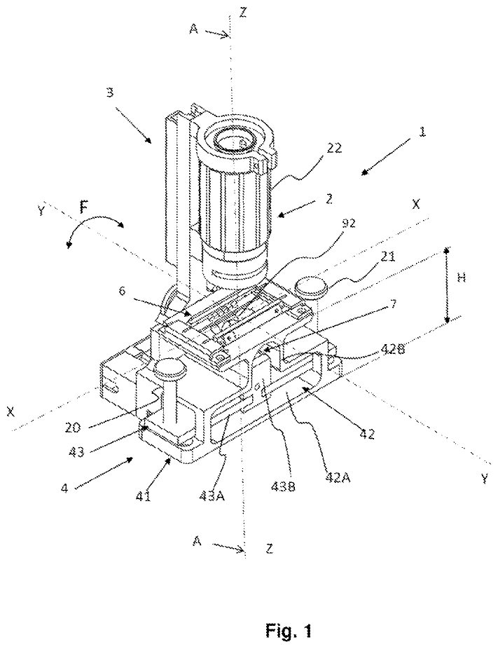

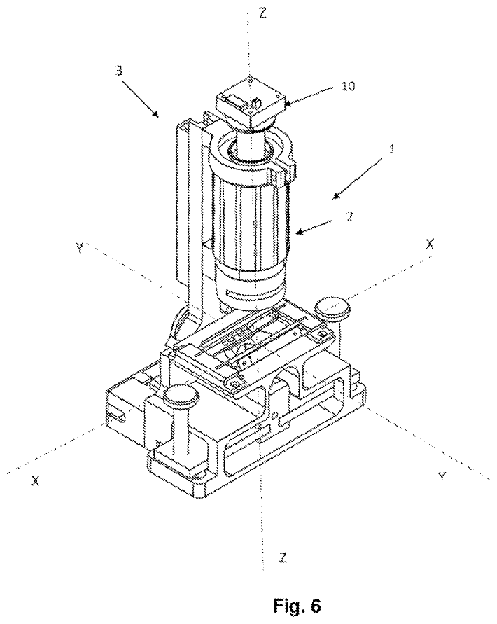

[FIG. 6] is a perspective view of the examining device of FIG. 1 equipped with a camera inserted in an optical microscope.

### DETAILED DESCRIPTION

In the various figures, the features designated by the same reference designate identical or similar features.

The terms “upper”, “lower” and their derivatives relate to locations of features or parts of features of the examination device when positioned in use.

In FIG. 1 of the present application, there is schematically shown a perspective view of a device 1 for examining a capillary area of the skin of a finger having a main axis. The device of the present disclosure is particularly suitable for examining a capillary area at the level of the nail of a finger.

The examining device is arranged in an orthonormal XYZ frame, the Z-Z axis being oriented in the vertical direction, and the plane (XY) corresponding to a horizontal plane.

The device comprises an optical microscope 2 mounted on a microscope arm 3 which extends along a vertical axis ZZ, a light source 6 for illuminating a capillary area, and a support 4. In FIG. 1, when the finger (not shown) is positioned under the microscope, the main axis of the finger is aligned along an axis YY.

As is illustrated in FIG. 6, the microscope 2 is equipped with a camera 10 which transmits the images to a display screen by appropriate wired or wireless means. The camera is inserted in the optical tube of the microscope.

The support 4 comprises a fixed base 41 having a cavity 42 with a profile in the form of an inverted “T”. More specifically, the cavity is formed by a first part 42A, the “T” base which extends in a plane (XY), and a second part 42B, the central web of the “T”, oriented along the axis Z-Z. Of course, the cavity is not limited only to this geometric shape.

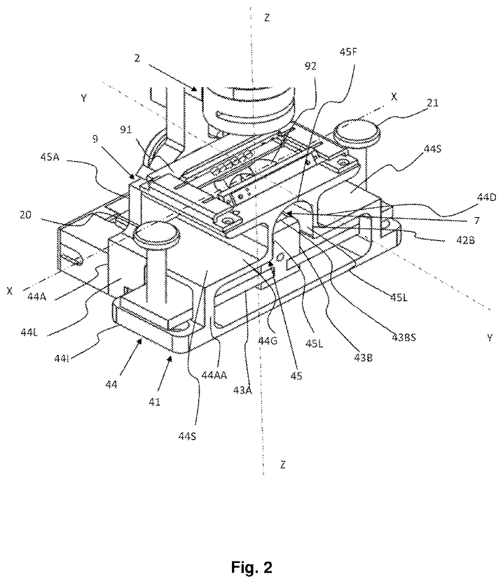

As is illustrated in FIGS. 1 and 2, the first part 42A of the cavity 42 is formed in a first component 44 having a substantially elongate parallelepipedal shape in the X-X direction. The first component 44 comprises two openworked side walls 44L, a rear wall 44A, an openworked front wall 44AA, an upper wall 44S and a lower wall 44I. The second part 42B of the cavity is formed in a second component 45 having a substantially elongate shape in the Y-Y direction. The second component 45 has an inverted U section with a bottom 45F arranged in the plane (XY) and two side branches 45L extending along the plane (YZ), and a rear wall 45A. The second component 45 is arranged substantially at the center of the upper wall 44S of the first component 44.

The fixed base 41 can be a one-piece component that has been machined or obtained by 3D printing. According to one variant, the fixed base 41 is formed from two separate components 44, 45 joined together to form the cavity shaped as an inverted T. The fixed base 41 further comprises stabilizing means in order to avoid any unwanted movement of the microscope during the examination. For this purpose, the first component 44 can be weighted. In one variant, the lower wall of the first component 44 can be equipped with adhesion means such as suction cups or adhesive elements.

The examining device comprises a movable component 43 having a profile in the shape of an inverted T, configured to be arranged in the cavity 42. The movable component 43 is not limited to this geometric shape. The movable component 43 comprises a first component 43A which extends in a plane (XY) and a second component 43B oriented along the axis Z-Z. The movable component 43 is arranged in the cavity 42 in such a way that the upper face 43BS of the second component 43B of the movable component 43 and the inner wall of the bottom 45F of the cavity 42 define a recess 7 intended to receive the finger. The upper face 43BS of the second component 43B of the movable component has a bearing surface on which the patient's finger will be positioned. The recess 7 has an elongate shape comprising a main axis oriented along the axis Y-Y. Thus, when the finger is inserted into the recess, the main axis of the finger and the main axis of the recess 7 are coincident. The second component 43B is held suspended within the cavity 42 by way of the first component 43A of the movable component. The latter has two ends joined to the ends of the lower wall 44I of the first component 44 of the fixed base 41 by clamping means 20, 21.

The two components 43A and 43B of the movable component are two separate components, and the second component 43B is mounted so as to be slidable relative to the first component 43A. Thus, it is possible to easily remove the second component 43B from the recess 7 for cleaning after the patient's finger has been examined.

Advantageously, the device comprises movement means 20, 21 which make it possible to move the movable component 43 in the cavity 42 between a first position and a second position. In the first position, the upper face 43BS of the second component 43B of the movable component 43 and the inner wall of the bottom 45F of the second component 45 of the fixed base 41 define a recess 7 having a dimension suitable for insertion of one of the fingers of the patient. In the second position, the upper face 43BS of the second component 43B of the movable component 43 and the inner wall of the bottom 45F of the second component 45 of the fixed base define a recess 7 having a dimension suitable for blocking the finger in the recess 7. The finger is immobilized against the inner wall of the bottom 45F of the second component 45 of the fixed base 45 without being compressed. The movement of the movable component 43 in the cavity 42 is carried out along the axis ZZ.

According to one aspect, the movement means are formed by the clamping means 20, 21 arranged on either side of the recess 7.

According to one variant, the movable component 43 can be moved by a pneumatic means. The pneumatic means comprise, for example, an inflatable cushion placed in the cavity 42A of the fixed base 41 of the device; the pressurization of the cushion could be triggered once the patient's finger is correctly positioned in the recess. It is adjusted automatically in order to have a level of compression for immobilizing the finger in the recess.

The recess 7 formed has a cross section substantially in the shape of an inverted “U”, with a substantially rounded bottom in order to be able to match the anatomical shape of the upper part of the finger when the latter is in a position bearing against the inner wall of the bottom 45F of the second component 45 of the fixed base 41.

The finger is thus immobilized in the recess 7 in the direction Z-Z, by its upper surface resting against the inner wall of the bottom 45F of the second component of the fixed base 45 and its lower surface resting against the upper face 43BS of the second component 43B of the movable component 43. In the direction X-X, the finger is blocked by the two inner walls of the two side branches 45L of the “U” of the recess 7 formed in the second component 45 of the fixed base 41, the finger thus being immobilized by its side surfaces.

The device further comprises a plate 9 which extends in the plane (XY) and which is fixed on the bottom 45F of the second component 45 of the fixed base. It has an upper face 91 oriented toward the microscope 2, with an opening 92 which communicates with the recess 7. Thus, the microscope 2 can observe the capillary area of the skin of the finger through the opening 92. Preferably, the plate 9 and the fixed base 41 form a single piece. According to one variant, the plate 9 is a separate component which is fixed to the bottom 45F of the fixed base 41 by conventional fixing means.

Advantageously, the upper wall 44S of the first component 44 of the fixed base 41 comprises two flanges 44G, 44D arranged relative to the recess 7 in such a way that the fingers juxtaposing the finger to be examined can be placed there without effort during the examination phase.

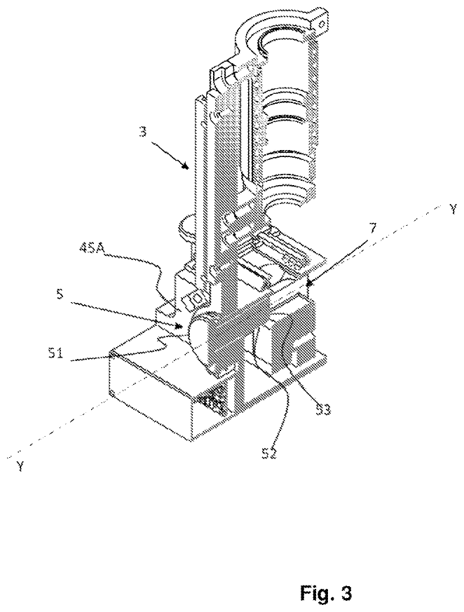

As is illustrated in FIG. 3, the device comprises a stop element 5 inserted into the recess 7 by the rear wall 45A of the second component 45 of the fixed base 41 via a hole. The stop element comprises a head 51 and an elongate body 52 inserted into the hole. The lateral end of the elongate body has a bearing surface 53 for the end of the finger when the latter is inserted into the recess 7. The stop element is formed by a clamping means, and the positioning of the stop element can be adjusted in the recess 7 by screwing, moving it in the direction Y-Y. The finger is thus also blocked in the direction Y-Y.

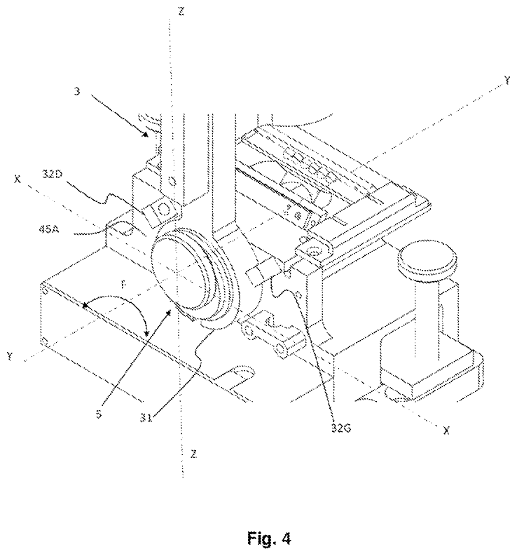

As is illustrated in FIG. 4, the examining device comprises a microscope arm 3 having a lower end 31 mounted to rotate on the support 4 via suitable pivot connection means. More specifically, the arm is moved in rotation in a plane (XZ) about an axis of rotation Y-Y which is the main axis of the finger, such that the microscope can image the capillary area from one edge of the finger to the other along the curvature of the finger, without moving the finger, which is immobilized in the recess 7 during the examination. The movement of the arm about the axis of rotation Y-Y can be continuous or step by step. Preferably, the arm can be moved between −15° and +15° relative to the axis Z-Z about the axis of rotation. The angular movement is represented by a double arrow F in FIG. 4. According to one aspect, the microscope can assume three positions in order to take three images: an image at the center, and an image at each edge of the finger. The device comprises two stops 32D, 32G arranged respectively to the right and to the left of the lower end 31 of the arm 3 and arranged on the rear wall 45A of the second component 45 of the fixed base 41 in order to fix the two angular positions of the arm on either side of the axis Z-Z. The two stops are arranged in such a way that the arm can move away from the central position by a maximum angle of between 10 and 15° on each side.

The rotation of the arm therefore always makes it possible to place the optical axis of the microscope perpendicularly with respect to the surface of the capillary area of the skin of the finger that is to be observed, this regardless of the curvature of the surface that is to be examined. In this way, it is possible to image both the center and the two edges of the cuticle without moving the finger to be examined.

The pivot connection means provided at the end 31 of the arm 3 are conventional means. They can comprise a rotation pin which protrudes from the rear wall 45A of the second component about which the lower end 31 of the arm is mounted in rotation. Standard mechanical markers arranged on the arm 3 and on the upper face 91 of the plate 9 allow the arm 3 to be positioned in a vertical position.

The microscope 2 is fixed to the arm 3, with the optical axis of the microscope extending parallel to the axis of the arm. As is shown in FIG. 1, the outer wall 22 of the microscope forms a focusing ring for the optical objective and the camera of the microscope. It is thus possible for the physician, using just one hand, to move the arm relative to the axis of rotation Y-Y to the desired angular position and to carry out the focusing of the microscope, which entails rotating the microscope about the axis Z-Z in order to obtain a vertical movement along the axis Z-Z of the optical objective.

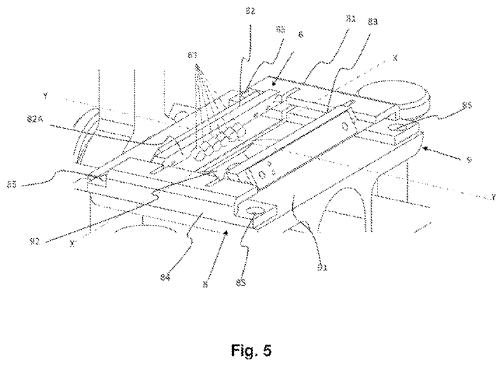

Referring to FIG. 5, the examining device comprises a light source 6 for illuminating the capillary area of the skin of the finger that is to be examined. The light source comprises, for example, a plurality of light-emitting diodes (LEDs) 61. According to one aspect, the LEDs comprise LEDs of various colors, for example blue and green so as to eliminate the red component. According to one preferred aspect, the LEDs emit white light. In this case, the light source comprises an optical band-pass filter which is arranged on the path of the incident optical beam emitted by the LEDs or in front of the optics of the microscope in order to eliminate the red component. The elimination of the red component advantageously makes it possible to enhance the contrast of the capillaries in the image.

According to one variant, the light source is a laser source suitable for carrying out analyses of marker fluorine in fluorescence microscopy. In general, the light source is suitable for performing any other type of marker analysis.

The light source comprises at least one row of aligned LEDs. The row extends along the axis X-X at an edge of the opening of the upper plate 9, such that the overall incident beam supplied by the LEDs is oriented in the direction of the main axis of the finger. The row of LEDs is arranged, with respect to the surface of the capillary area of the finger, in such a way as to generate low-angled illumination on the surface of the examination area, in order to suppress parasitic reflections. To increase the illumination and obtain homogeneous illumination, a second row of LEDs can be arranged parallel to the first row and on an opposite edge of the opening 92.

Preferably, the device comprises a polarizer and an analyzer which are arranged on the optical path between the light source and the microscope in order to eliminate parasitic reflections. By way of example, the polarizer is arranged between the beam, emitted by the LEDs, and the examination area. The analyzer is arranged between the beam, reflected from the examination area, and the microscope.

Referring to FIG. 5, the device comprises a support 8 intended to receive the two rows of LEDs 61. The support is composed of four bars 81, 82, 83, 84 forming a frame that is intended to frame the opening 92 when the support 8 is fixed on the upper face 91 of the plate 9, with two bars 81, 84 oriented along the axis Y-Y and two bars 82, 83 oriented along the axis X-X. Each of the bars 82, 83 oriented along the axis X-X has a substantially inclined surface 82A oriented toward the capillary area for examination and provided with a row of LEDs. The support 8 is fixed to the plate 9 using four conventional fixing means 85.

According to one aspect, the height (H), represented by a double arrow in FIG. 1, of the base is defined such that the wrist of the hand to be examined is placed on the surface on which the fixed base is placed. Thus, this ergonomic position makes it possible not to induce tension in the finger to be examined, which tension can distort the results of the capillary examination.

The examining device of the present disclosure is configured to receive other examination modules such as modules for measuring sugar level, oxygen, heart rate or lactate.

### INDUSTRIAL APPLICATION

The present disclosure may be applied to any field where it is necessary to perform an examination by capillaroscopy. The present disclosure is particularly suitable for examining the capillary area at the cuticle.

The present disclosure is not limited to the examples described above; features of the examples illustrated can in particular be combined with each other within variants that are not illustrated.

What is claimed is:

1. A device for examining a capillary area of the skin of a finger, said device comprising:
an optical microscope;
a light source for illuminating the capillary area;
a support comprising a recess for receiving said finger, said recess comprising an opening positioned opposite the microscope;
said microscope being mounted to rotate on the support about a main axis of the finger, so as to scan the capillary area from one edge of said finger to the other;
immobilizing means being configured in such a way as to adjust the dimension of the recess relative to the shape of the finger, in order to immobilize the finger in said recess without exerting pressure on the finger.

2. The device as claimed in claim 1, in which the immobilizing means are configured to immobilize said finger in the recess without compressing it in the three directions X, Y and Z.

3. The device as claimed in claim 1, in which said immobilizing means are configured to immobilize said finger by its upper and lower surfaces and by its side surfaces.

4. The device as claimed in claim 1, in which the microscope is mounted on an arm, one end of said arm being mounted to rotate on the support about the main axis of the finger, such that the microscope can perform a scan of the capillary area from one edge of said finger to the other.

5. The device as claimed in claim 1, in which the support comprises a fixed base having a cavity, a movable component arranged in the cavity (42), said movable component being adapted to be moved between a first position, in which it forms within the space of the cavity a recess having a dimension adapted for insertion of the finger, and a second position, in which it forms within the space of the cavity a recess having a dimension adapted to immobilize the finger in the recess without exerting pressure on the finger.

6. The device as claimed in claim 1, in which the recess has a surface intended to match the shape of the finger when the finger is immobilized in the recess.

7. The device as claimed in claim 1, further comprising a stop element arranged in the recess in such a way as to form a bearing surface for one end of the finger along the main axis when the finger is positioned in the recess.

8. The device as claimed in claim 1, in which the light source is arranged in such a way as provide low-angled illumination on the capillary area.

9. The device as claimed in claim 1, further comprising a polarizer and an analyzer which are arranged on the optical path between the light source and the microscope in order to eliminated parasitic reflections.

10. The device as claimed in claim 1, further comprising at least one optical band-pass filter intended to eliminate a red component of the image obtained by the microscope.
